(19) United States
(12) Patent Application Publication
(10) Pub. No.: US 2026/0000893 A1
(43) Pub. Date: Jan. 1, 2026

(54) HEART PRELOAD MODULATION

(71) Applicant: EDWARDS LIFESCIENCES CORPORATION, Irvine, CA (US)

(72) Inventor: Arnold Cruz Tuason, Claremont, CA (US)

(21) Appl. No.: 19/323,103

(22) Filed: Sep. 9, 2025

Related U.S. Application Data
(63) Continuation of application No. PCT/US2024/019144, filed on Mar. 8, 2024.
(60) Provisional application No. 63/489,898, filed on Mar. 13, 2023.

(51) Int. Cl. A61N 1/36

(52) U.S. Cl. CPC A61N 1/36114; A61N 1/36139

(57) ABSTRACT

A medical therapy system includes an electrical stimulation device having a at least one electrode that stimulates a splanchnic nerve, a sensor device for providing a sensor measurement indicative of a heart preload, and a controller configured to, in response to the sensor measurement, generate a control signal for causing the electrical stimulation device to deliver an electric stimulation signal to the splanchnic nerve.

### CROSS REFERENCE TO RELATED APPLICATION(S)

This application is a continuation of International Patent Application No. PCT/US2024/019144, filed Mar. 8, 2024, which claims the benefit of U.S. Provisional Patent Application Ser. No. 63/489,898, filed Mar. 13, 2023, the complete disclosures of which are hereby incorporated by reference herein in their entireties for all purposes.

### BACKGROUND

Modulation of heart preload can benefit patients with heart preload that is too high or too low. Heart preload modulation can be useful in various clinical settings, including in the emergency department, intensive care unit and/or perioperative settings.

### SUMMARY

Described herein are methods and devices relating to modulation of a heart preload. A medical therapy system configured to provide heart preload modulation can comprise a sensor device configured to provide sensor measurements, an electrical stimulation device configured to provide electrical stimulation to one or more nerves, a generator configured to generate one or more electrical stimulation signals for delivery by the electrical stimulation device, and a controller can be configured to generate one or more control signals based on the sensor measurement. In some instances, in response to receiving the sensor measurement, the controller can be configured to generate the one or more control signals. The one or more control signals can be transmitted to the generator such that the one or more electrical stimulation signals can be generated by the generator. The one or more electrical stimulation signals can be subsequently transmitted to the electrical stimulation device for delivery to the one or more nerves. In some instances, the one or more nerves can comprise one or more splanchnic nerves. In some instances, the medical therapy system can be configured to automatically modulate the heart preload, including modulating the heart preload without further operator input after an initial trigger and/or activation input.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages, and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1A is a block diagram representation of a medical therapy system configured to modulate a heart preload in accordance with one or more examples. FIG. 1B shows the medical therapy system described with reference to FIG. 1A in accordance with one or more examples.

FIG. 2 is a graph showing changes in a heart preload as a function of time due to heart preload modulation in accordance with one or more examples.

FIG. 3 is a diagram showing various regions of the splanchnic nerves that can be electrically stimulated to modulate heart preload in accordance with one or more examples.

FIG. 4 shows a side cut-away view of a thoracic cavity and positions of electrical stimulation devices within the intercostal veins and/or intercostal arteries for electrical stimulation of splanchnic nerves within the thoracic cavity, in accordance with one or more examples.

FIGS. 5 is a diagram showing ganglion and post-ganglion locations that can be electrically stimulated for modulating heart preload and positions of electrical stimulation devices for electrical stimulation of the splanchnic nerves, in accordance with one or more examples.

FIG. 6 is a process flow diagram of a process for modulating a heart preload in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

To support cardiac function, a need to treat patients whose heart preload is either too high or too low exists in various clinical settings, including in the emergency department, intensive care units, or and/or in a perioperative environment. Modulation of heart preload can benefit patients with heart preload that is too high or too low. For example, patients suffering from acute pulmonary edema due to volume overload or redistribution can have high heart preload. These patients need to reduce their preload. Typically reduction of preload can be achieved by administering diuretics, including for patients suffering from acute pulmonary edema. Patients needing an increase in the heart preload, including patients suffering from cardiogenic shock, can typically be intravenously injected with a bolus of fluid to raise the heart preload. Perioperative patients may suffer from either too much or too little preload. In some cases, preload can be affected by mechanical ventilation, changes in sympathetic tone associated with intra-operative general anesthesia and/or hormonal changes in the postoperative setting. Patients sensitive to these acute changes in preload, such as those with pulmonary hypertension, may decompensate due to acute changes in preload.

Described herein are methods and devices relating to modulating a heart preload, including automated modulation of the heart preload. A medical therapy system configured to provide heart preload modulation can comprise a sensor device, an electrical stimulation device, a generator and a controller. The sensor device can be configured to provide a sensor measurement that is indicative of a heart preload. In some instances, the sensor measurement can be used to determine whether the heart preload is within a target range. The electrical stimulation device can be configured to provide electrical stimulation to one or more nerves. The generator can be configured to generate one or more electrical stimulation signals for delivery by the electrical stimulation device. The controller can be configured to generate a control signal, based on the sensor measurement, for generating the one or more electrical stimulation signals. For example, in response to the sensor measurement, the controller can be configured to generate the control signal. The control signal can be transmitted to the generator such that the electrical stimulation signals can be generated by the generator, and subsequently transmitted to the electrical stimulation device for delivery to the one or more nerves. In some instances, the one or more nerves can comprise one or more splanchnic nerves.

The medical therapy system can be configured to stimulate or block the one or more splanchnic nerves to increase or decrease the heart preload, respectively. Stimulation of the one or more splanchnic nerves can result in vasoconstriction of splanchnic blood vessels, thereby increasing the heart preload. Blocking the one or more splanchnic nerves can provide vasodilation of splanchnic blood vessels, thereby decreasing the heart preload. In some instances, the medical therapy system can be configured to modulate the heart preload completely automatically. For example, the medical therapy system can be configured to modulate the heart preload with no further interference from an operator after an initial trigger and/or activation input from the operator.

The splanchnic venous reservoir can provide a mechanism for modulating the heart preload. The splanchnic venous reservoir can serve as a reservoir of blood. The splanchnic venous reservoir can have up to about 20% of the total blood volume. At least a portion of this reservoir can be placed into and out of the systemic stressed circulation. A method of manipulating the splanchnic reservoir for the purposes of controlling preload can have an immediate impact. Manipulation of the splanchnic venous reservoir can be used instead of or in combination with other interventions, such as intravenous injection of fluid boluses and/or diuretic drugs.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

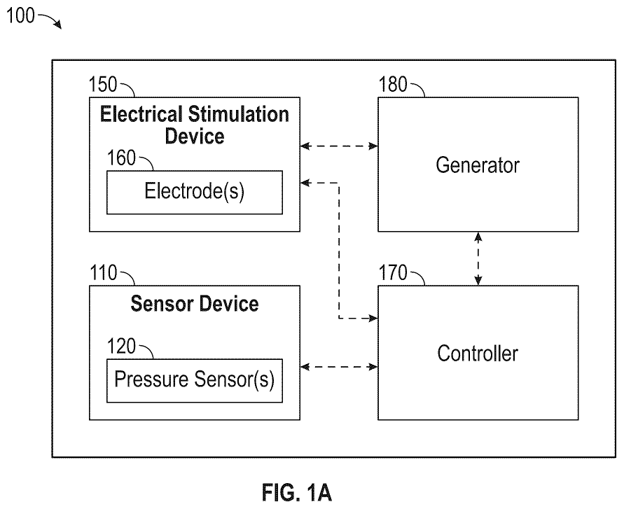

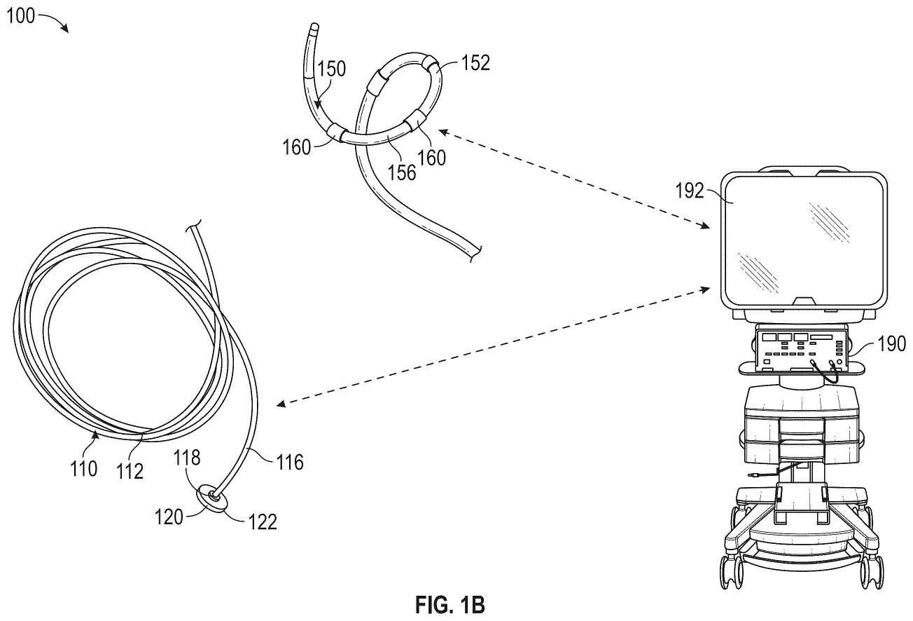

FIG. 1A shows a block diagram of an example of a medical therapy system 100 configured to modulate a heart preload. In some instances, the medical therapy system 100 can provide automatic modulation of the heart preload, such that a target heart preload is achieved. The medical therapy system 100 can comprise a sensor device 110, an electrical stimulation device 150, a controller 170, and a generator 180. The sensor device 110 can comprise one or more sensors, such as one or more pressure sensors 120, each configured to provide a sensor measurement that can be indicative of a heart preload. The electrical stimulation device 150 can comprise one or more electrodes 160 to deliver electrical stimulation to one or more nerves. The generator 180 can be configured to generate one or more electrical stimulation signals for transmission to the electrical stimulation device 150 such that the electrical stimulation can be delivered to the nerves. The controller 170 can be configured to generate one or more control signals to cause the electrical stimulation device 150 to deliver the electrical stimulation to the one or more nerves, so as to modulate the heart preload. The one or more control signals can be generated based at least in part on the one or more sensor measurements provided by the sensor device 110.

The controller 170 can be in direct or indirect communication with the sensor device 110, the electrical stimulation device 150 and/or the generator 180. For example, the sensor device 110 and the controller 170 can be in direct or indirect communication with one another such that sensor measurements made by the sensor device 110, including the one or more pressure sensors 120, can be communicated to the controller 170. The generator 180 and the controller 170 can be in direct or indirect communication with one another such that control signals generated by the controller 170 for generating the electrical stimulation signals can be communicated to the generator 180. The electrical stimulation device 150 and the generator 180 can be in direct or indirect communication with one another such that the electrical stimulation signals generated by the generator 180 can be transmitted to the electrical stimulation device 150.

In some instances, the one or more nerves can comprise one or more splanchnic nerves. The medical therapy system 100 can be configured to stimulate or block the one or more splanchnic nerves to increase or decrease the heart preload, respectively. Applying electrical stimulation to the splanchnic nerves can stimulate or block the splanchnic nerves. Stimulation of the one or more splanchnic nerves can result in vasoconstriction of splanchnic blood vessels, thereby increasing the heart preload. Blocking the one or more splanchnic nerves can provide vasodilation of splanchnic blood vessels, thereby decreasing the heart preload.

The controller 170 may include certain control circuitry configured to perform certain of the functionality described herein. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in instances in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry. The control circuitry may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry may be entirely locally maintained/disposed or may be remotely located at least in part (e.g., communicatively coupled indirectly via a local area network and/or a wide area network). Any of the control circuitry may be configured to perform any aspect(s) of the various processes disclosed herein.

FIG. 1B shows an example of the medical therapy system 100 described with reference to FIG. 1A. As described herein, the medical therapy system 100 can comprise the sensor device 110, the electrical stimulation device 150, the controller 170 (not shown), and the generator 180 (not shown). The sensor device 110 can comprise at least a portion configured to be positioned within the vasculature of the patient to provide the one or more sensor measurements that can be indicative of the heart preload. The electrical stimulation device 150 can comprise at least a portion configured to be positioned within the patient, including the vasculature of the patient, to provide electrical stimulation to the one or more nerves, including the splanchnic nerves. The controller 170 and/or the generator 180 can be externally housed within a housing 190. In some instances, the medical therapy system 100 can comprise a user interface 192 configured to display various monitored parameters and/or receive input from an operator.

In some instances, the sensor device 110 can comprise a pressure sensor 120 configured to provide measurements of a blood pressure. The sensor measurement can comprise a measurement of the blood pressure, including a venous pressure. In some instances, the sensor measurement provided by the sensor device 110 can be used to determine a heart preload of the patient. In some alternative instances, the sensor measurement itself and/or another value determined using the sensor measurement can serve as a proxy to determine whether the heart preload is within a normal range. For example, the controller 170 can determine whether the heart preload is within a normal range using the blood pressure measurement such that the controller 170 can generate a control signal to cause the electrical stimulation device 150 to electrically stimulate the one or more nerves to modulate the heart preload if the pressure measurement indicates that the heart preload is outside of the normal range. In some instances, the pressure sensor 120 can be configured to measure a central venous pressure. The sensor device 110 can comprise a catheter 112 and the pressure sensor 120 can be associated with a distal portion 116 of the catheter 112. The pressure sensor 120 can be coupled to, disposed around and/or extending from, the distal portion 116 of the catheter 112, including a distal end 118 of the catheter 112. The pressure sensor 120 can be configured to measure the central venous pressure at any number of different time intervals. The sensor device 110 can be in communication with the controller 170 such that measurements made by the pressure sensor 120 can be communicated to the controller 170.

In some instances, the sensor device 110 can comprise an anchor 122, such as an inflatable balloon, configured to stabilize the position of the pressure sensor 120. In some instances, at least a portion of the catheter 112 can be advanced along a venous access route. A trans-subclavian or trans-jugular approach can be used. For example, a portion of the catheter 112 can be advanced through a subclavian or jugular vein to position the distal portion 116 of the catheter 112 at a target location to facilitate measurement by the pressure sensor 120 of the central venous pressure. In some instances, the pressure sensor 120 can be positioned at a location within a superior vena cava and/or right atrium for monitoring the central venous pressure. FIG. 1B shows the inflatable balloon in an inflated state. For example, the inflatable balloon can be inflated after the distal portion 116 of the catheter 112 is desirably positioned to maintain the location of the pressure sensor 120.

The electrical stimulation device 150 can comprise one or more electrodes 160 configured to provide stimulation to the one or more nerves. The electrical stimulation device 150 can comprise a catheter 152 and the one or more electrodes 160 can be associated with a distal portion 156 of the catheter 152. The one or more electrodes 160 can be coupled to, disposed around, and/or extending from, the distal portion 156 of the catheter 152. At least a portion of the catheter 152 can be advanced into the vasculature to position the distal portion 156 at a target location within a blood vessel. The one or more electrodes 160 can be configured to be in contact with respective wall portions of the blood vessel at the target location such that one or more electrical stimulation signals transmitted to the one or more electrodes 160 can be delivered to the blood vessel wall portions. The electrical stimulation signals can travel through the blood vessel wall portions to the one or more splanchnic nerves proximate and/or adjacent to the blood vessel wall portions. In some instances, electrical stimulation signals can travel from the electrodes 160 through the blood vessel wall portions to the one or more splanchnic nerves in contact with the blood vessel wall portions. In some instances, the electrical stimulation device 150 can comprise a plurality of electrodes 160 configured to electrically stimulate the one or more splanchnic nerves.

The sensor device 110 and the electrical stimulation device 150 can be distinct devices. Alternatively, in some instances, at least a portion of the sensor device 110 and the electrical stimulation device 150 can form the same device. For example, the catheter 112 of the sensor device 110 and the catheter 152 of the electrical stimulation device 150 can extend from a common proximal handle. In some instances, a proximal portion of each of the catheter 112 and the catheter 152 can extend along a common axis, including being housed within a common outer catheter and/or forming a portion of the same catheter. In some instances, more than one electrical stimulation devices 150 may be used to provide electrical stimulation of more than one target site and/or area. In some instances, the one or more electrical stimulation devices 150 and the sensor device 110 can form the same device. The one or more electrical stimulation devices 150 and the sensor device 110 can extend from a common proximal handle, and/or comprise at least a portion extending along a common axis, being housed within a common outer catheter and/or forming a portion of the same catheter.

In some alternative instances, an electrical stimulation device can comprise a stimulation needle configured to provide percutaneous access to a target site. For example, the stimulation needle can be configured to be percutaneously positioned to the target site. In some instances, the stimulation needle can be inserted into the patient through a posterior or dorsal access site such that a distal portion of the stimulation needle can be at the target site.

Electrical stimulation delivered to the one or more splanchnic nerves can stimulate or block the splanchnic nerves. In some instances, electrical stimulation delivered to the one or more splanchnic nerves can stimulate the splanchnic nerves to provide vasoconstriction of splanchnic blood vessels. Vasoconstriction of the splanchnic blood vessels can place blood from the splanchnic venous reservoir into systemic circulation, and thereby increase the heart preload. Electrical stimulation for stimulating the splanchnic nerves can be delivered if the sensor measurement indicates the heart preload is below a target range. In some instances, electrical stimulation delivered to the one or more splanchnic nerves can block the splanchnic nerve to provide vasodilation of splanchnic blood vessels. Vasodilation of the splanchnic blood vessels can move blood in systemic circulation into the splanchnic venous reservoir, and thereby decrease the heart preload. Electrical stimulation for blocking the splanchnic nerves can be delivered if the sensor measurement indicates the heart preload is above the target range.

The controller 170 can be in direct or indirect communication with the sensor device 110 and the electrical stimulation device 150 to facilitate modulation of the heart preload. The controller 170 can be configured to receive the sensor measurement and determine if the sensor measurement indicates whether the heart preload is within the target range. The controller 170 can be configured to generate a control signal to cause the electrical stimulation device 150 to deliver an electrical stimulation signal to the one or more splanchnic nerves if the sensor measurement indicates the heart preload is outside of the target range. The generator 180 can be configured to generate electrical stimulation signals used for electrically stimulating the one or more splanchnic nerves. The controller 170 can be in direct or indirect communication with the generator 180 such that the generator 180 can generate the electrical stimulation signal in response to the control signal from the controller 170.

In some instances, the generator 180 can be configured to generate electrical stimulation signals configured for temporary and/or reversible stimulation and/or blocking of the splanchnic nerves. In some instances, temporary and/or reversible stimulation or blocking can comprise stimulating or blocking the splanchnic nerve for a finite period of time such that the splanchnic nerves return to an unstimulated or unblocked state after the finite period of time. In some instances, temporary and/or reversible stimulation or blocking can comprise stimulating or blocking the splanchnic nerve for a predetermined period of time. In some instances, temporary and/or reversible stimulation or blocking can comprise stimulating or blocking the splanchnic nerve for the duration or substantially the duration while the electrical stimulation signal is applied. The generator 180 can be in direct or indirect communication with the electrical stimulation device 150 such that the electrical stimulation signals generated by the generator 180 can be transmitted to the electrical stimulation device 150 for delivery to the target tissue through the one or more electrodes 160. In some instances, the generator 180 can be configured to generate electrical stimulation signals having a frequency of about 3 Hertz (Hz) to about 20 Hertz (Hz), such as to cause vasoconstriction of splanchnic blood vessels, including temporary and/or reversible vasoconstriction of the splanchnic blood vessels. In some instances, the generator 180 can be configured to generate electrical stimulation signals having a frequency of about 1 kilo Hertz (kHz) to about 30 kilo Hertz (kHz), so as to cause vasodilation of splanchnic blood vessels, including temporary and/or reversible vasodilation of the splanchnic blood vessels. In some instances, the electrical stimulation signals can be delivered intermittently, such as to prevent or reduce any permanent nerve damage and/or improve control in change to the heart preload. For example, a period of electrical stimulation can be followed by a period of no electrical stimulation, which can be repeated to provide desired stimulation or blocking of the splanchnic nerves. The periods of electrical stimulation and/or no electrical stimulation can be regular and/or irregular.

In some instances, the medical therapy system 100 can be configured to select a frequency for one or more electrical stimulation signals. In some instances, a sweep of frequencies can be performed, such as within one or more of the ranges as described herein, while taking the sensor measurements. A frequency can be selected to provide desired nerve stimulation or nerve block based on, including but not limited to, patient anatomy and/or patient health status. For example, using the sensor measurements, such as the pressure measurements, a frequency can be selected to provide the desired nerve block or nerve stimulation. In some instances, the frequency can be used for subsequent nerve block or nerve stimulation of the patient. In some instances, a frequency sweep can be performed for each nerve block or nerve stimulation. In some instances, the frequency sweep can be performed by a separate device configured to be in communication with the medical therapy system 100.

In some instances, the control signal generated by the controller 170 can be configured to be transmitted to the generator 180 for instructing the generator 180 regarding one or more electrical stimulation signals the generator 180 is to generate. The generator 180 can then generate one or more electrical stimulation signals based on the control signal. For example, the generator 180 can comprise an input configured to receive control signals from the controller 170. In some instances, the generator 180 can comprise an input configured to be coupled to an output of the controller 170. The generator 180 can comprise an output configured to be coupled to an input of the electrical stimulation device 150. In some instances, in response to receiving the control signal, the generator 180 can be configured to generate the one or more electrical stimulation signals. The electrical stimulation signals can be transmitted to the electrical stimulation device 150 for delivery to the target tissue.

In some instances, the medical therapy system 100 can be configured to provide continuous modulation of the heart preload until a preset target is achieved, such as until the heart preload is within the target range, and/or for a predetermined period, including a period set by an operator. For example, the sensor device 110 can be configured to provide continuous sensing of the blood pressure, including at any number of time intervals (e.g., every second, every minute), until an input is received to stop the measurements and/or until a preset period of time has elapsed. Each measurement made by the sensor device 110 can be transmit to the controller 170 such that the controller 170 can determine whether the heart preload is within range. If the sensor measurement indicates that the heart preload is outside the target range, the controller 170 can be configured to generate one or more control signals for the generator 180 to generate one or more electrical stimulation signals for delivery to the target tissue by electrical stimulation device 150. This process can be continued, such as making the sensor measurement and delivering the electrical stimulation to the target tissue until the heart preload is within range. In some instances, the sensor device 110 can be configured to continue making sensor measurements while the heart preload is within range. The controller 170 can continue to use the sensor measurements to determine whether the heart preload is within range. In some instances, while the heart preload is determined to be within range, no control signals for electrical stimulation signals are generated by the controller 170.

In some instances, the controller 170 can generate one or more control signals for generating one or more electrical stimulation signals to be delivered to the target tissue while the heart preload is determined to be within range but is predicted to become outside of the range. For example, the one or more control signals can be generated by the controller 170 if one or more subsequent sensor measurements will indicate that the heart preload that is outside of the range, such as the next sensor measurement. The control signals can be generated based on a predicted value of the heart preload. If the heart preload is predicted to be outside of the target range, the control signals can be generated to modulate the heart preload before the heart preload goes outside of the target range. Modulation of the heart preload can be performed before the heart preload is outside of the target range. In some instances, the predicted heart preload can be determined based at least in part on prior one or more sensor measurements and/or a change in prior sensor measurements. For example, a determination of whether the heart preload will become outside of the range can be based at least on a sensor measurement and/or a heart preload value determined using the sensor measurement, and a rate of change of the sensor measurement and/or a rate of change of the heart preload determined using sensor measurements.

In some instances, the controller 170 can be configured adjust electrical stimulation signals generated by the generator 180 based on the sensor measurements, for example generating an adjustment control signal configured to modify the electrical stimulation signal. For example, a second control signal for electrical stimulation of the splanchnic nerve, in response to receiving a second sensor measurement, where the second control signal can be different from a previous control signal. For example, the second control signal can cause the electrical stimulation device to deliver a stimulation signal to the splanchnic nerve different from a previous stimulation signal delivered to the splanchnic nerve, such as for adjusting the stimulation of the splanchnic nerve.

The medical therapy system 100 can provide partially and/or completely automatic modulation of the heart preload. In some instances, the medical therapy system 100 can be configured to provide a completely automated modulation of the heart preload. For example, after insertion of the sensor device 110 and electrical stimulation device 150, in response to a trigger and/or activation input from an operator, the medical therapy system 100 can be configured to begin modulating the heart preload. The medical therapy system 100 can subsequently modulate the heart preload without further input from the operator. In response to the trigger and/or activation input from the operator, the medical therapy system 100 can be configured to begin to make sensor measurements. In some instances, the medical therapy system 100 can begin to make sensor measurements in response to the medical therapy system 100 being turned on and/or activated. The medical therapy system 100 can then stimulate or block the one or more splanchnic nerves based whether the sensor measurements indicate the heart preload is below or above the target range, respectively. In some instances, medical therapy system 100 can then stimulate or block the one or more splanchnic nerves based whether the sensor measurements indicate the heart preload will be below or above the target range, respectively. The stimulating or blocking can be performed without further input from the operator. For example, in response to receiving the sensor measurements and without further input from the operator, the controller 170 can determine whether the sensor measurement indicates that the heart preload is below or above the target range, and provide a control signal for stimulating or blocking the splanchnic nerves, respectively. The control signal can be provided to the generator 180 by the controller 170 for generating the electrical stimulation signal to stimulate or block the splanchnic nerves. The generator 180 can generate the electrical stimulation signals without interference from the operator so as to facilitate providing automated modulation of the heart preload. The electrical stimulation signal generated by the generator 180 can be transmitted to the electrical stimulation device 150, which can deliver the electrical stimulation signal to the splanchnic nerves. Making the sensor measurements by the sensor device, determining whether the heart preload is and/or will be within range and generating the control signal by the controller 170, generating the electrical stimulation signal by the generator 180 and delivering the electrical stimulation signal to the splanchnic nerves by the electrical stimulation device 150 can be performed without further input from the operator, such as after the initial trigger provided by the operator.

In some instances, the generator 180 can be integrated with the electrical stimulation device 150. For example, control signals generated by the controller 170 can be provided to the electrical stimulation device 150 for generating and delivering of the electrical stimulation signals. Alternatively, in some instances, the generator 180 can be integrated with the controller 170, such that control signals from the controller 170 is provided to the generator 180 integrated therewith.

In the alternative or combination, the medical therapy system 100 can include a sensor device 110 comprising a sensor to provide measures of at least one of a right atrial pressure, pulmonary arterial pressure, and pulmonary capillary wedge pressure. A central venous pressure, right atrial pressure, pulmonary arterial pressure and/or pulmonary capillary wedge pressure can be used to determine whether the heart preload is within a normal range.

In the alternative or combination, a sensor device can comprise at least one of a blood oxygen level sensor, heart rate variability sensor, and baroreflex sensitivity sensor. Sensor measurements made by the blood oxygen level sensor, heart rate variability sensor, and/or baroreflex sensitivity sensor can be used in place of or in combination with a pressure sensor for determining whether a heart preload is within the target range. For example, blood oxygen level can be measured using pulse oximetry, and/or blood sampling. In the alternative or combination, afferent nerves can be target locations for therapy, including for example, carotid body baroreceptors and/or aortic arch baroreceptors.

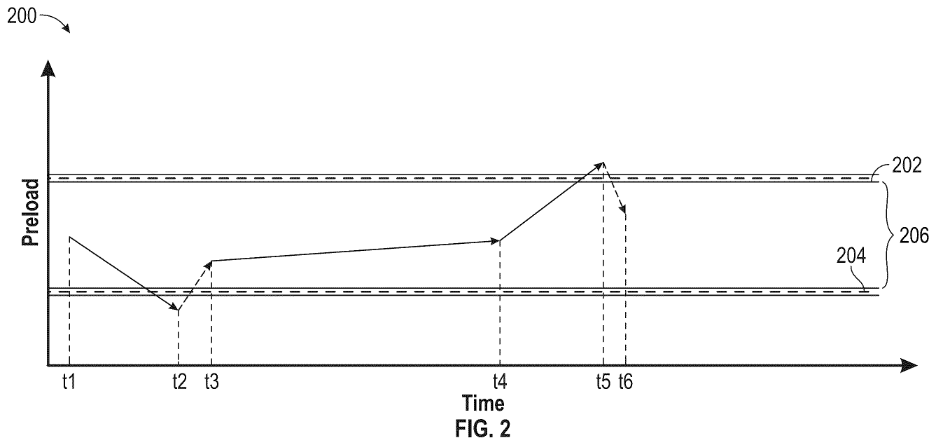

FIG. 2 is a graph 200 showing an example of changes in a heart preload as a function of time due to heart preload modulation. The y-axis of the graph is the heart preload, for example measured in millimeters of mercury (mmHg) and the x-axis is time, for example expressed in seconds (sec). A medical therapy system, including the medical therapy system 100 described with reference to FIGS. 1A and 1B, can be configured to maintain the heart preload within a normal range. A sensor device can be configured to make sensor measurements used to determine the heart preload, such as by a controller. For example, sensor measurements can be used to determine and/or estimate the heart preload such that a determination can be made regarding whether the heart preload is above an upper preload value 202 or below a lower preload value 204. Sensor measurements can be used to determine and/or estimate the heart preload to determine if the heart preload is within a target range 206. For example, a first sensor measurement can be made at time t1. A determination can be made indicating that the heart preload is within the target range 206. In response to determining that the preload is within range, no electrical stimulation signal is delivered to the splanchnic nerves. In some instances, a controller can receive the first sensor measurement. The controller can determine whether or not the heart preload is within range based at least in part on the first sensor measurement. In response to a determination that the heart preload is within range, the controller does not generate a control signal for electrical stimulation. A second sensor measurement can be made at time t2. The controller can receive the second measurement. The second sensor measurement can indicate that the heart preload is outside of the target range 206, such as being below the lower preload value 204. For example, the controller can determine that the heart preload is not within range based at least in part on the second sensor measurement. In response to the second sensor measurement indicating that the heart preload is below the lower preload value 204, the controller of the medical therapy system can generate a control signal for increasing heart preload. For example, the controller can generate a control signal for providing an electrical stimulation signal that stimulates one or more splanchnic nerves to cause vasoconstriction of splanchnic blood vessels and increase the heart preload. Subsequent sensor measurements can be made. For example, a third and a fourth sensor measurement can be made at time t3 and time t4. The controller can receive the subsequent sensor measurements. A determination can be made, such as by the controller, indicating that the heart preload is within the target range 206. In response to determining that the preload is within range, no electrical stimulation signal is delivered to the splanchnic nerves after the third and fourth sensor measurements. The controller does not generate a control signal for electrical stimulation. A fifth sensor measurement can be made at time t5. The fifth sensor measurement can indicate that, such as determined by the controller, the heart preload is outside of the target range 206, such as being above the upper preload value 202. In response to the fifth sensor measurement indicating the heart preload is above the upper preload value 202, the controller of the medical therapy system can generate a control signal for decreasing heart preload. For example, the controller can generate a control signal for providing an electrical stimulation signal that blocks one or more splanchnic nerves to cause vasodilation of splanchnic blood vessels and decrease the heart preload. Subsequent sensor measurements can be made, such as a sixth sensor measurement at time t₆. In response to determining the preload is within range based on the sixth sensor measurement, no electrical stimulation signal is delivered to the splanchnic nerves. Monitoring of the heart preload can continue. In some instances, sensor measurements can be made for a time period set by an operator, until an input from the operator to stop the measurements, and/or until a preset parameter is achieved. Although FIG. 2 is described as including sensor measurements at certain points in time, it will be understood that additional measurements can be made in between any two of the described points in time, including at regular time intervals between the two points in time.

In some instances, the target preload range can be set by an operator. In some instances, the target range can be a normal range as understood by one skilled in the art. The target range can depend at least in part on the type of sensor measurement taken, such as the type of pressure being monitored. In some instances, the normal range can be between about 2 millimeters of mercury (mmHg) and 6 millimeters of mercury (mmHg). In some instances, the normal range can be between about 6 millimeters of mercury (mmHg) and 12 millimeters of mercury (mmHg).

In some instances, one or more electrical stimulations signals can be delivered to the splanchnic nerves prior to the heart preload becoming outside of the target range 206. Electrical stimulation can be applied pre-emptively such that the heart preload is maintained or substantially maintained within the target range 206. In some instances, a rate of change of the heart preload, and/or a rate of change of a sensor measurement, can be used to determine whether an electrical stimulation signal should be applied. For example, the measured heart preload, including a measured heart preload as determined based at least in part by a sensor measurement, and rate of change of the heart preload can be used to determine if the heart preload will be outside of the target range 206, for example at the next measurement time. In some instances, a sensor measurement and rate of change of the sensor measurement can be used. If it is determined that the heart preload will be outside of the target range 206, one or more electrical stimulation signals can be applied to prevent the heart preload from becoming above the upper preload value 202 or below the lower preload value 204.

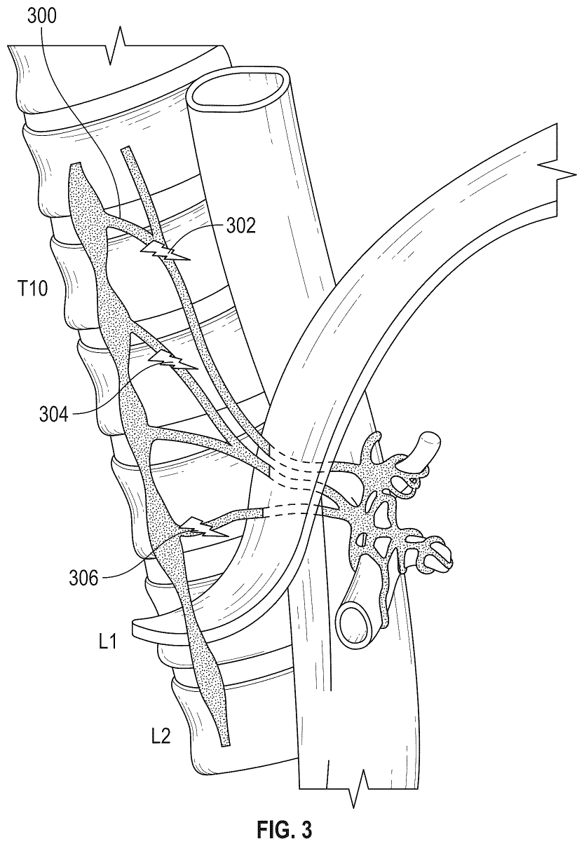

Referring to FIG. 3, a diagram is shown of various regions of the splanchnic nerves 300 that can be electrically stimulated to modulate heart preload. Electrical stimulation can be delivered to one or more regions of the splanchnic nerves 300, including for example thoracic splanchnic nerves. The electrical stimulation can be delivered using a medical therapy system described herein, such as the medical therapy system 100 described with reference to FIGS. 1A and 1B. In some instances, the electrical stimulation can be delivered to a location on a greater splanchnic nerve 302. In some instances, the electrical stimulation can be delivered to a location on a lesser splanchnic nerve 304. In some instances, the electrical stimulation can be delivered to a location on a least splanchnic nerve 306.

In some instances, electrical stimulation of one or more regions of the greater splanchnic nerve 302, lesser splanchnic nerve 304 and/or least splanchnic nerve 306 can provide desired modulation of the heart preload without causing undesired adverse outcomes. In some instances, applying electrical stimulation to the greater splanchnic nerve 302 can provide greater therapeutic impact than applying electrical stimulation to the lesser splanchnic nerve 304 and/or the least splanchnic nerve 306. In some instances, increased therapeutic impact may result in increased risk for adverse reactions. One or more locations of the splanchnic nerves can be electrically stimulated while avoiding or reducing any resulting adverse reactions.

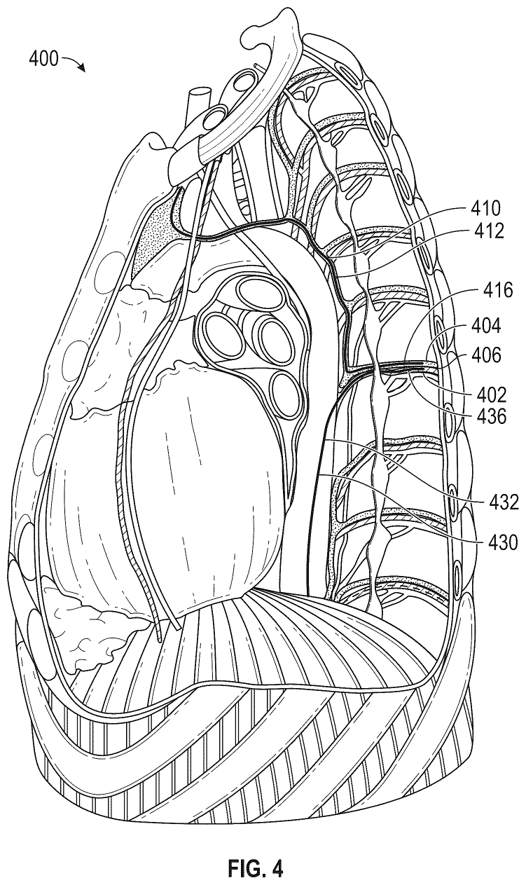

As described herein, delivery of electrical stimulation to one or more nerves can comprise transvascular delivery. FIG. 4 shows a side cut-away view of certain anatomical features in a thoracic cavity 400. Portions of the splanchnic nerves 402 can be proximate and/or adjacent to intercostal veins 404 and/or intercostal arteries 406. In some instances, portions of the splanchnic nerves 402, including thoracic splanchnic nerves, can run alongside intercostal veins 404 and/or intercostal arteries 406. Delivery of electrical stimulation to a target portion of the splanchnic nerves can be performed using a venous blood vessel or an arterial blood vessel. The electrical stimulation can be delivered using a medical therapy system described herein, such as the medical therapy system 100 described with reference to FIGS. 1A and 1B.

In some instances, delivery of electrical stimulation to a target portion of the splanchnic nerves can be performed through a venous blood vessel. An example placement of a portion of an electrical stimulation device 410 into venous blood vessels is shown in FIG. 4. For example, the electrical stimulation device 410 can deliver electrical stimulation to a wall portion of a venous blood vessel such that the electrical stimulation can be transmitted to one or more splanchnic nerves in direct or indirect contact with the venous blood vessel wall portion. At least a portion of a catheter 412 of the electrical stimulation device 410 can be inserted into a venous access site and advanced along a venous route. One or more electrodes of the electrical stimulation device 410 disposed on a distal portion 416 of the catheter 412 can be positioned in contact with the wall portion of the venous blood vessel for delivery of the electrical stimulation. In some instances, the target portion of the splanchnic nerves can be accessed using an intercostal vein 404. For example, trans-jugular or trans-subclavian approach can be applied to insert a portion of the electrical stimulation device 410, such as a portion of the catheter 412, into the jugular vein or subclavian vein, respectively. The portion of the electrical stimulation device 410, including the portion of the catheter 412, can be advanced through the jugular vein or the subclavian vein to be positioned within the intercostal vein 404, including a posterior intercostal vein. In some instances, the portion of the electrical stimulation device 410, including the portion of the catheter 412, can be advanced through the jugular vein or the subclavian vein, and a brachiocephalic vein to be positioned within the intercostal vein 404.

The one or more electrodes of the electrical stimulation device 410 can be positioned to be in contact with respective wall portions of the intercostal vein 404 such that electrical stimulation delivered to the electrodes can be transferred to the wall portions of the intercostal vein 404, and thereby delivered to target splanchnic nerve portions proximate and/or adjacent to the wall portions.

In alternative or in combination, delivery of electrical stimulation to a target portion of the splanchnic nerves can be performed through an arterial blood vessel. An example placement of a portion of an electrical stimulation device 430 into arterial blood vessels is shown in FIG. 4. The electrical stimulation device 430 can comprise a catheter 432 having at least a portion configured to be positioned through an arterial route. In some instances, the catheter 432 of the electrical stimulation device 430 can be advanced into an arterial access site and advanced into the aorta. A distal portion 436 of the catheter 432 comprising electrodes disposed thereon can be advanced through a portion of the aorta and into a target arterial blood vessel. Electrodes of the electrical stimulation device 430 can be positioned at a target location within the arterial blood vessel, including an intercostal artery 406. For example, an electrical stimulation device 430 can deliver electrical stimulation to a wall portion of an arterial blood vessel such that the electrical stimulation can be transmitted to one or more splanchnic nerves in direct or indirect contact with the arterial blood vessel wall portion, including a portion of a wall portion of the intercostal artery 406. One or more electrodes of the electrical stimulation device 430 can be positioned in contact with the wall portion of the arterial blood vessel, such as the wall portion of the intercostal artery 406, for delivery of the electrical stimulation.

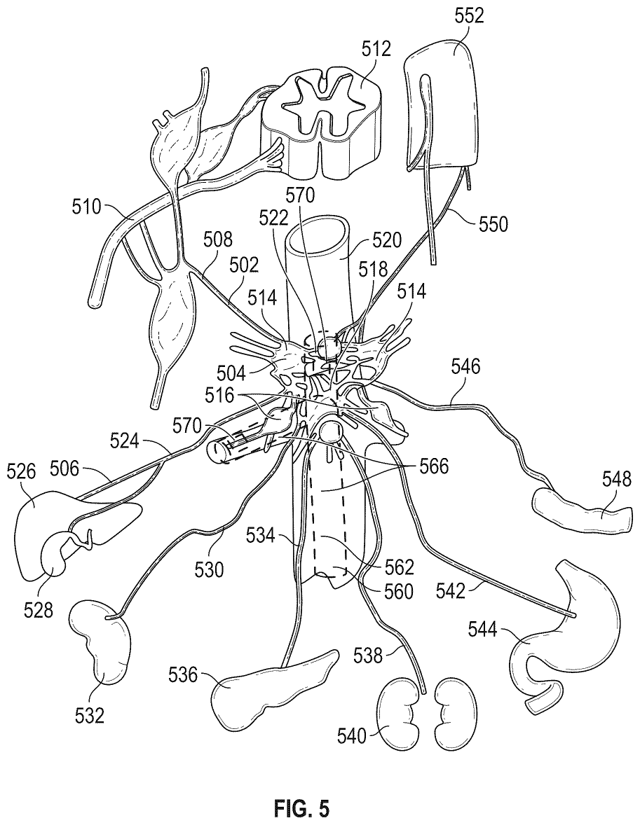

Referring to FIG. 5, in some instances, electrical stimulation can be delivered to a pre-ganglion location 502, a ganglion location 504, and/or a post-ganglion location 506. The electrical stimulation can be delivered using a medical therapy system described herein, such as the medical therapy system 100 described with reference to FIGS. 1A and 1B. In some instances, electrical stimulation can be delivered to a pre-ganglion location 502 comprising one or more thoracic splanchnic nerves 508. The thoracic splanchnic nerves 508 can originate from spinal nerves 510 that interact with the spinal cord 512. Targeting the thoracic splanchnic nerves 508 originating from the spinal nerves 510 and descending into the celiac ganglions can have the advantage of allowing for a venous access procedure. As described herein, electrical stimulation of thoracic splanchnic nerves 508 can be delivered using the intercostal veins.

In some instances, the electrical stimulation can be delivered to a ganglion location 504 located at the junction of the aorta 520 and splanchnic arteries 522. In some instances, the electrical stimulation can be delivered to one or more of the celiac ganglions 514. In some instances, the electrical stimulation can be delivered to one or more of the aorticorenal ganglions 516. In some instances, the electrical stimulation can be delivered to a superior mesenteric ganglions 518. Targeting a ganglion location 504, such as the celiac ganglion 514, may advantageously provide greater therapeutic impact, as the ganglion location 504 can be a junction of multiple nerves. For example, a ganglion location 504, such as the celiac ganglion 514, can advantageously provide greater therapeutic impact than by targeting a thoracic splanchnic nerve, including a greater, lesser and/or least splanchnic nerve.

In some instances, the electrical stimulation can be delivered to a post-ganglion location 506, such as to post-ganglion nerves that enervate the arteries 522. In instances where limiting therapy is desired, targeting the post-ganglion nerves can advantageously limit therapy to organs being fed by the targeted artery. In some instances, electrical stimulation can be delivered to one or more post-ganglion nerves at a post-ganglion location 524 that enervates arteries supplying blood to the liver 526 and/or gallbladder 528. In some instances, electrical stimulation can be delivered to one or more post-ganglion nerves at a post-ganglion location 530 that enervates arteries supplying blood to the spleen 532. In some instances, electrical stimulation can be delivered to one or more post-ganglion nerves at a post-ganglion location 534 that enervates arteries supplying blood to the pancreas 536. In some instances, electrical stimulation can be delivered to one or more post-ganglion nerves at a post-ganglion location 538 that enervates arteries supplying blood to the kidneys 540. In some instances, electrical stimulation can be delivered to one or more post-ganglion nerves at a post-ganglion location 542 that enervates arteries supplying blood to the stomach 544. In some instances, electrical stimulation can be delivered to one or more post-ganglion nerves at a post-ganglion location 546 that enervates arteries supplying blood to the large and/or small bowel 548. In some instances, electrical stimulation can be delivered to one or more post-ganglion nerves at a post-ganglion location 550 that enervates arteries supplying blood to the esophagus 552.

In some instances, access for delivering the electrical stimulation to a ganglion location 504 and/or a post-ganglion location 506 can be through an artery. For example, an electrical stimulation device can be inserted through an arterial insertion site and advanced along one or more arterial blood vessels, including the aorta 520, to position one or more electrodes of the electrical stimulation device at a target location to stimulate and/or block nerves at the target ganglion location 504 and/or post-ganglion location 506.

As shown in FIG. 5, in some instances, at least a portion of an electrical stimulation device 560 can be positioned into the aorta 520 to provide electrical stimulation at a ganglion location 504. For example, a distal portion 566 of a catheter 562 of the electrical stimulation device 560 can comprise one or more electrodes 570 that can be positioned at a target location in the aorta 520. FIG. 5 only shows the distal portion 566 of the catheter 562 for simplicity. The one or more electrodes 570 can provide electrical stimulation of a wall portion of the aorta 520, thereby providing electrical stimulation to nerves at the ganglion location 504 proximate and/or adjacent to the wall portion. In some instances, the ganglion location 504 can be over and/or in contact with the wall portion of the aorta 520 receiving the electrical stimulation.

In some instances, the distal portion 566 of the catheter 562 of the electrical stimulation device 560 can be positioned at a target location in an arterial blood vessel that directly or indirectly branches from the aorta 520, for example to provide electrical stimulation to nerves at a post-ganglion location 506. The one or more electrodes 570 disposed on the distal portion 566 of the catheter 562 can provide electrical stimulation of a wall portion of the arterial blood vessel directly or indirectly branching from the aorta 520, thereby providing electrical stimulation to nerves at the post-ganglion location 506 proximate and/or adjacent to the wall portion. In some instances, the post-ganglion location 506 can be over and/or in contact with the wall portion of the arterial blood vessel receiving the electrical stimulation.

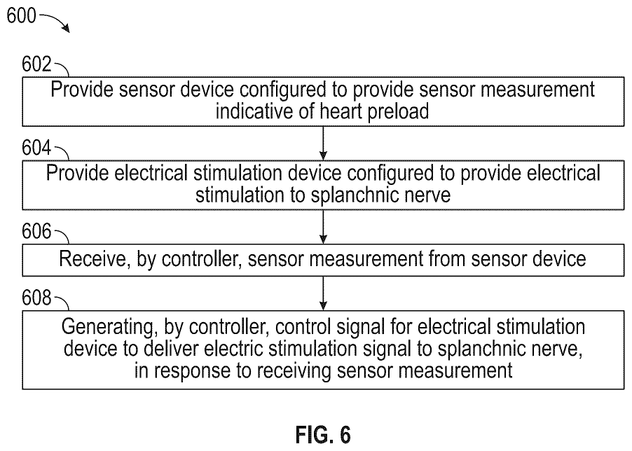

FIG. 6 is a process flow diagram of an example of a process 600 for delivery of medical therapy to a target nerve. The process 600 can be performed using a medical therapy system as described herein, including medical therapy system 100 described with reference to FIGS. 1A and 1B. In block 602, the process can involve providing a sensor device configured to provide a sensor measurement indicative of a heart preload. In block 604, the process can involve providing an electrical stimulation device configured to provide electrical stimulation to a splanchnic nerve. In block 606, the process can involve receiving, by a controller, a sensor measurement from the sensor device. In block 608, the process can involve generating, by the controller, a control signal for the electrical stimulation device to deliver an electric stimulation signal to the splanchnic nerve, in response to receiving the sensor measurement. In some instances, a plurality of control signals can be generated in response to the sensor measurement. In some instances, a plurality of electrical stimulation signals can be delivered to the splanchnic nerve in response to the sensor measurement.

In some instances, the process can involve determining, by the controller, if the sensor measurement indicates that the heart preload is within range and/or will remain within range. For example, the process can involve determining by the controller whether the heart preload will remain within range at one or more subsequent measurement times. The controller can generate the control signal if the sensor measurement indicates that the heart preload is outside the range or will be outside of the range, for example a normal range for the heart preload. In some instances, the process can involve providing a generator configured to generate an electrical stimulation signal. The control signal generated by the controller can be provided to the generator to cause the generator to generate the electrical stimulation signal. For example, the process can involve transmitting of the control signal generated by the controller to the generator. The generator can then generate, in response to the control signal, the electrical stimulation signal, and transmit the electrical stimulation signal to the electrical stimulation device. In some instances, the process can involve generating by the generator an electrical stimulation signal having one or more parameters based at least in part on the control signal. The electrical stimulation device can deliver the electrical stimulation signal to a target tissue, including a target blood vessel wall portion and/or a target splanchnic nerve.

In some instances, the process can involve temporarily and/or reversibly stimulating or blocking the splanchnic nerve. For example, in response to receiving the sensor measurement, the controller can generate a control signal for temporarily and/or reversibly stimulating or blocking the splanchnic nerve. The process can involve transmitting of the control signal for temporarily and/or reversibly stimulating or blocking the splanchnic nerve generated by the controller to the generator. The generator can then generate, in response to the control signal, the electrical stimulation signal, and transmit the electrical stimulation signal to the electrical stimulation device for temporarily and/or reversibly stimulating the target tissue.

In some instances, temporarily and/or reversibly stimulating the splanchnic nerve can comprise stimulating the splanchnic nerve to provide vasoconstriction of splanchnic blood vessels and increase the heart preload, such as if the sensor measurement indicates the heart preload is and/or will be below the target range. In some instances, temporarily and/or reversibly stimulating the splanchnic nerve can comprise blocking the splanchnic nerve to provide vasodilation of splanchnic blood vessels and decrease the heart preload, such as if the sensor measurement indicates the heart preload is and/or will be above the target range.

In some instances, temporarily and/or reversibly stimulating the splanchnic nerve can comprise providing an electrical stimulation signal to an electrode of the stimulation device, the electrical stimulation signal having a frequency of about 3 Hertz (Hz) to about 20 Hertz (Hz) to cause vasoconstriction of splanchnic blood vessels, including about 5 Hertz (Hz) to about 15 Hertz (Hz). In some instances, temporarily and/or reversibly stimulating the splanchnic nerve can comprise providing an electrical stimulation signal to an electrode of the stimulation component, the electrical stimulation signal having a frequency of about 1 kilo Hertz (kHz) to about 30 kilo Hertz (kHz) to cause vasodilation of splanchnic blood vessels, including about 5 kilo Hertz (kHz) to about 20 kilo Hertz (kHz).

In some instances, the process can involve providing continuous modulation of the heart preload. The sensor device can continuously make sensor measurements, including at regular and/or predetermined time intervals. For example, the process can involve receiving, by the controller, additional sensor measurements from the sensor device. In response to the additional sensor measurements, additional control signals can be provided by the controller for electrical stimulation of the splanchnic nerve by the electrical stimulation device. For example, in response to receiving each sensor measurement, the controller can determine if the sensor measurement indicates the heart preload is and/or will be within range, and generate a control signal for electrical stimulation of the splanchnic nerve if the sensor measurement indicates the heart preload is and/or will not be within range. In some instances, the sensor device can continuously make sensor measurements for a predetermined period of time, until a sensor measurement indicates the heart preload is within range, and/or until an input is received from an operator to stop the sensor measurements.

In some instances, the controller can be configured to adjust control signals based on the sensor measurements to provide a target heart preload. In some instances, the process can involve receiving, by the controller, a second sensor measurement from the sensor device, and generating, by the controller, a second control signal for electrical stimulation of the splanchnic nerve, in response to receiving the second sensor measurement. The second control signal can be different from a previous control signal. For example, the second control signal can cause the electrical stimulation device to deliver a stimulation signal to the splanchnic nerve different from a previous stimulation signal delivered to the splanchnic nerve, such as for adjusting the stimulation of the splanchnic nerve.

In some instances, generating, by the controller, a control signal for the electrical stimulation device can comprise generating a control signal for the electrical stimulation device to deliver an electric stimulation signal to at least one of a greater splanchnic, lesser splanchnic nerve, and least splanchnic nerve. In some instances, generating, by the controller, a control signal for the electrical stimulation device can comprise generating a control signal for the electrical stimulation device to deliver an electric stimulation signal to at least one of a pre-ganglion location, ganglion location and post-ganglion location. In some instances, generating, by the controller, a control signal for the electrical stimulation device can comprise generating a control signal for the electrical stimulation device to deliver an electric stimulation signal to at least one of a celiac ganglion, superior mesenteric ganglion and aorticorenal ganglion.

In some instances, electrically stimulating the splanchnic nerve can comprise transvascularly stimulating the splanchnic nerve through a wall portion of a blood vessel. For example, the process can involve positioning at least a portion of the electrical stimulation device, including one or more electrodes of the electrical stimulation device, into a blood vessel, such as through a venous or arterial access site, and electrically stimulating a wall portion of the blood vessel. Electrical stimulation applied to the blood vessel wall portion can be transmitted to the target splanchnic nerve. Electrically stimulating the splanchnic nerve can comprise electrically stimulating a blood vessel wall portion proximate and/or adjacent to the target splanchnic nerve, including a blood vessel wall portion in direct contact with the target splanchnic nerve.

In some instances, the process 600 can be automated, including partially or completely automated. In some instances, the process 600 can be completely automated such that the heart preload can be modulated after an initial trigger and/or activation input is received from an operator. For example, making the sensor measurements by the sensor device, determining whether the heart preload is within range and generating the control signal by the controller, communicating the control signal to the generator, generating the electrical stimulation signal by the generator, and transmitting the electrical stimulation signal to the electrical stimulation device, and delivering the electrical stimulation signal to the splanchnic nerves by the electrical stimulation device can be performed without further input from the operator.

### Description of Additional Examples

Provided below is a list of examples, each of which may include aspects of any of the other examples disclosed herein. Furthermore, aspects of any example described above may be implemented in any of the numbered examples provided below.

Example 1: A medical therapy system comprising an electrical stimulation device including a plurality of electrodes configured to electrically stimulate a splanchnic nerve, a sensor device configured to provide a sensor measurement indicative of a heart preload, and a controller configured to, in response to the sensor measurement, generate a control signal configured to cause the electrical stimulation device to deliver an electrical stimulation signal to the splanchnic nerve.

Example 2: The system of any example herein, in particular example 1, wherein the controller is configured to generate the control signal in response to receiving the sensor measurement if the sensor measurement indicates the heart preload is not within range.

Example 3: The system of any example herein, in particular example 1 or 2, wherein the controller, in response to receiving the sensor measurement, is configured to determine if the heart preload is within range.

Example 4: The system of any example herein, in particular examples 1 to 3, wherein the controller is configured to generate an adjustment control signal configured to modify the electrical stimulation signal for temporarily stimulating or temporarily blocking the splanchnic nerve.

Example 5: The system of any example herein, in particular examples 1 to 4, wherein the controller, in response to receiving the sensor measurement, is configured to generate a control signal for causing the electrical stimulation device to deliver the electrical stimulation signal for temporarily stimulating the splanchnic nerve if the sensor measurement indicates the heart preload is below the range, or deliver the electrical stimulation signal for temporarily blocking the splanchnic nerve if the sensor measurement indicates the heart preload is above the range.

Example 6: The system of any example herein, in particular example 5, further comprising a generator, wherein the controller is configured to transmit the control signal to the generator to generate the electrical stimulation signal for temporarily stimulating the splanchnic nerve if the sensor measurement indicates the heart preload is below the range, or generate the electrical stimulation signal for temporarily blocking the splanchnic nerve if the sensor measurement indicates the heart preload is above the range.

Example 7: The system of any example herein, in particular examples 1 to 6, wherein the electrical stimulation device is configured to deliver the electrical stimulation signal to a greater splanchnic nerve.

Example 8: The system of any example herein, in particular examples 1 to 7, wherein the electrical stimulation device is configured to deliver the electrical stimulation signal to a lesser splanchnic nerve.

Example 9: The system of any example herein, in particular examples 1 to 8, wherein the electrical stimulation device is configured to deliver the electrical stimulation signal to a least splanchnic nerve.

Example 10: The system of any example herein, in particular examples 1 to 9, wherein the electrical stimulation device is configured to deliver the electrical stimulation signal to a splanchnic nerve at a pre-ganglion location.

Example 11: The system of any example herein, in particular examples 1 to 10, wherein the electrical stimulation device is configured to deliver the electrical stimulation signal to a splanchnic nerve at a post-ganglion location.

Example 12: The system of any example herein, in particular examples 1 to 11, wherein the electrical stimulation device is configured to deliver the electrical stimulation signal to a ganglion.

Example 13: The system of any example herein, in particular examples 1 to 12, wherein the sensor device comprises a pressure sensor configured to measure blood pressure.

Example 14: The system of any example herein, in particular example 13, wherein the pressure sensor is configured to measure venous pressure.

Example 15: The system of any example herein, in particular example 13 or 14, wherein the pressure sensor is configured to measure at least one of a central venous pressure, right atrial pressure, pulmonary arterial pressure, and pulmonary capillary wedge pressure.

Example 16: The system of any example herein, in particular examples 1 to 15, wherein the sensor device comprises at least one of a blood oxygen level sensor, heart rate variability sensor, and baroreflex sensitivity sensor.

Example 17: The system of any example herein, in particular examples 1 to 16, wherein the plurality of electrodes is configured to be positioned within a blood vessel and in contact with a blood vessel wall portion in contact with the splanchnic nerve.

Example 18: An automated method of delivering medical therapy comprising providing a sensor device configured to provide a sensor measurement indicative of a heart preload, providing an electrical stimulation device configured to provide electrical stimulation to a splanchnic nerve, receiving, by a controller, a sensor measurement from the sensor device, and generating, by the controller, a control signal for the electrical stimulation device to deliver an electric stimulation signal to the splanchnic nerve, in response to receiving the sensor measurement.

Example 19: The method of any example herein, in particular example 18, further comprising determining, by the controller, if the sensor measurement indicates the heart preload is within range.

Example 20: The method of any example herein, in particular example 18 or 19, further comprising stimulating the splanchnic nerve, in response to receiving the sensor measurement.

Example 21: The method of any example herein, in particular example 20, wherein stimulating the splanchnic nerve comprises stimulating the splanchnic nerve to provide vasoconstriction of splanchnic blood vessels and increase the heart preload, if the sensor measurement indicates the heart preload is below a range, or blocking the splanchnic nerve to provide vasodilation of splanchnic blood vessels and decrease the heart preload, if the sensor measurement indicates the heart preload is above the range.

Example 22: The method of any example herein, in particular example 20 or 21, wherein stimulating the splanchnic nerve comprises transvascularly stimulating the splanchnic nerve through a wall portion of a blood vessel.

Example 23: The method of any example herein, in particular examples 20 to 22, wherein stimulating the splanchnic nerve comprises providing an electrical stimulation signal to an electrode of the stimulation device having a frequency of about 3 Hertz (Hz) to about 20 Hertz (Hz) to cause vasoconstriction of splanchnic blood vessels, or providing an electrical stimulation signal to an electrode of the stimulation device having a frequency of about 1 kilo Hertz (kHz) to about 30 kilo Hertz (kHz) to cause vasodilation of splanchnic blood vessels.

Example 24: The method of any example herein, in particular examples 18 to 23, further comprising providing additional control signals to the electrical stimulation device for electrical stimulation of the splanchnic nerve, and receiving, by the controller, additional sensor measurements from the sensor device, until an additional sensor measurement indicates the heart preload is within range.

Example 25: The method of any example herein, in particular examples 18 to 24, further comprising receiving, by the controller, a second sensor measurement from the sensor device, and providing, by the controller, a second control signal to the electrical stimulation device, in response to receiving the second sensor measurement, to deliver an electrical stimulation signal to the splanchnic nerve different from a previous electrical stimulation signal for adjusting the electrical stimulation of the splanchnic nerve.

Example 26: The method of any example herein, in particular examples 18 to 25, wherein generating the control signal comprises generating a control signal for stimulating a greater splanchnic nerve.

Example 27: The method of any example herein, in particular examples 18 to 26, wherein generating the control signal comprises generating a control signal for stimulating a lesser splanchnic nerve.

Example 28: The method of any example herein, in particular examples 18 to 27, wherein generating the control signal comprises generating a control signal for stimulating a least splanchnic nerve.

Example 29: The method of any example herein, in particular examples 18 to 28, wherein generating the control signal comprises generating a control signal for stimulating a splanchnic nerve at a pre-ganglion location.

Example 30: The method of any example herein, in particular examples 18 to 29, wherein generating the control signal comprises generating a control signal for stimulating a splanchnic nerve at a post-ganglion location.

Example 31: The method of any example herein, in particular examples 18 to 30, wherein generating the control signal comprises generating a control signal for stimulating a celiac ganglion.

Example 32: The method of any example herein, in particular examples 18 to 31, wherein generating the control signal comprises generating a control signal for stimulating an aorticorenal ganglion.

The above method(s) can be performed on a living animal or on a simulation, such as on a cadaver, cadaver heart, anthropomorphic ghost, simulator (e.g., with body parts, heart, tissue, etc. being simulated).

Example 33: A medical therapy system comprising control circuitry communicatively coupled to an electrical stimulation device and a sensor device, the control circuitry being configured to generate a control signal based at least in part on a sensor measurement from the sensor device, and the control signal being configured to cause to the electrical stimulation device to deliver an electrical stimulation signal to the splanchnic nerve to modulate a heart preload.

Example 34: The system of any example herein, in particular example 33, wherein the control signal generated by the control circuitry is configured to cause a generator to generate an electrical stimulation signal for delivery to the splanchnic nerve by the electrical stimulation device.

Example 35: The system of any example herein, in particular example 33 or 34, wherein the control circuitry is configured to determine, using the sensor measurement, whether the heart preload is within range, and generate the control signal if the sensor measurement indicates the heart preload is not within range.

Example 36: The system of any example herein, in particular examples 33 to 35, wherein the control circuitry, in response to receiving the sensor measurement, is configured to generate a control signal for causing the electrical stimulation device to deliver the electrical stimulation signal for stimulating the splanchnic nerve if the sensor measurement indicates the heart preload is below the range, and deliver the electrical stimulation signal for blocking the splanchnic nerve if the sensor measurement indicates the heart preload is above the range.

Example 37: The system of any example herein, in particular examples 33 to 36, wherein the sensor device comprises a sensor for measuring at least one of a central venous pressure, right atrial pressure, pulmonary arterial pressure, and pulmonary capillary wedge pressure, and the control circuitry is configured to determine whether the heart preload is within range using at least one of the central venous pressure, right atrial pressure, pulmonary arterial pressure, and pulmonary capillary wedge pressure.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, clements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, clement, etc. may be either X, Y or Z. Thus, such conjunctive language is not generally intended to imply that certain examples require at least one of X, at least one of Y and at least one of Z to cach be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other instance(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated. comparative and/or quantitative terms, such as “less,” “more,” “greater,” and the like. are intended to encompass the concepts of equality. For example, “less” can mean not only “less” in the strictest mathematical sense, but also, “less than or equal to.”

What is claimed is:

1. A method for modulating cardiac preload, the method comprising:
providing a sensor device positioned to sense venous pressures and provide a sensor measurement indicative of a heart preload;
providing an electrical stimulation device adjacent to a splanchnic nerve including at least one of a greater splanchnic nerve, a lesser splanchnic nerve, and a least splanchnic nerve;
receiving, by a controller, the sensor measurement from the sensor device, wherein the controller is in direct or indirect communication with the sensor device;
in response to the sensor measurement indicating that the heart preload is below a pressure range, generating, by the controller, a first control signal to cause the electrical stimulation device to temporarily stimulate the splanchnic nerve at a first frequency to cause vasoconstriction of splanchnic blood vessels; and
in response to the sensor measurement indicating that the heart preload is above the pressure range, generating, by the controller, a second control signal to cause the electrical stimulation device to temporarily stimulate the splanchnic nerve at a second frequency to cause vasodilation of splanchnic blood vessels.

2. The method of claim 1, wherein the electrical stimulation device stimulates at least one of a splanchnic nerve at a pre-ganglion location, a ganglion location, and a post-ganglion.

3. The method of claim 1, wherein the sensor device measures at least one of a central venous pressure, right atrial pressure, pulmonary arterial pressure, and pulmonary capillary wedge pressure.

4. The method of claim 1, wherein the sensor device comprises at least one of a blood oxygen level sensor, heart rate variability sensor, and baroreflex sensitivity sensor.

5. The method of claim 1, further comprising, in response to the sensor measurement indicating that the heart preload is within the pressure range, not stimulating the splanchnic nerve.

6. The method of claim 1, wherein the electrical stimulation device is positioned within a blood vessel and in contact with a blood vessel wall portion in contact with the splanchnic nerve.

7. The method of claim 6, wherein the electrical stimulation device stimulates the splanchnic nerve through the blood vessel wall portion.

8. The method of claim 1, wherein the sensor device is coupled to the electrical stimulation device.

9. An automated method of delivering medical therapy, the method comprising:
providing a sensor device positioned to provide a sensor measurement indicative of a heart preload;
providing an electrical stimulation device adjacent to a splanchnic nerve, the electrical stimulation device comprising a plurality of electrodes;
receiving, by a controller, the sensor measurement from the sensor device, wherein the controller is in direct or indirect communication with the sensor device;
determining, by the controller, a rate of change of the heart preload based at least in part on the sensor measurement;
determining, by the controller, an estimated future heart preload based on the rate of change of the heart preload;
in response to the sensor measurement indicating that the estimated future heart preload is below a pressure range, generating, by the controller, a first control signal to cause the electrical stimulation device to stimulate the splanchnic nerve at a first frequency to cause vasoconstriction of splanchnic blood vessels; and
in response to the sensor measurement indicating that the estimated future heart preload is above the pressure range, generating, by the controller, a second control signal to cause the electrical stimulation device to stimulate the splanchnic nerve at a second frequency to cause vasodilation of splanchnic blood vessels.

10. The method of claim 9, wherein the electrical stimulation device stimulates the splanchnic nerve transvascularly through a wall portion of a blood vessel.

11. The method of claim 9, wherein stimulating the splanchnic nerve comprises:
providing the first electrical stimulation signal to an electrode of the electric stimulation device having a frequency of 3 Hertz (Hz) to 20 Hz to cause vasoconstriction of splanchnic blood vessels; or
providing the second electrical stimulation signal to an electrode of the electrical stimulation device having a frequency of 1 kilo Hertz (kHz) to 30 kHz to cause vasodilation of splanchnic blood vessels.

12. The method of claim 9, wherein the electrical stimulation device stimulates at least one of a splanchnic nerve at a pre-ganglion location, a ganglion location, and a post-ganglion.

13. The method of claim 9, wherein the sensor device measures at least one of a central venous pressure, right atrial pressure, pulmonary arterial pressure, and pulmonary capillary wedge pressure.

14. The method of claim 9, wherein the sensor device comprises at least one of a blood oxygen level sensor, heart rate variability sensor, and baroreflex sensitivity sensor.

15. The method of claim 9, wherein the sensor device is coupled to the electrical stimulation device.

16. A method for modulating cardiac preload, the method comprising:
providing a sensor device positioned to provide a first sensor measurement and a second sensor measurement indicative of a heart preload;
providing an electrical stimulation device adjacent to a splanchnic nerve, the electrical stimulation device comprising a plurality of electrodes;
receiving, by a controller, the first sensor measurement and the second sensor measurement from the sensor device, wherein the controller is in direct or indirect communication with the sensor device;
determining, by the controller, a rate of change of heart preload based on the first sensor measurement and the second sensor measurement;
determining, by the controller, a predicted heart preload based on the rate of change of the heart preload;
in response to the predicted heart preload indicating that heart preload will be below a pressure range, generating, by the controller, a first control signal to cause the electrical stimulation device to temporarily stimulate the splanchnic nerve; and
in response to the predicted heart preload indicating that heart preload will be above the pressure range, generating, by the controller, a second control signal to cause the electrical stimulation device to temporarily block the splanchnic nerve.

17. The method of claim 16, wherein the electrical stimulation device stimulates or blocks at least one of a greater splanchnic nerve, a lesser splanchnic nerve and a least splanchnic nerve.

18. The method of claim 16, wherein the electrical stimulation device is positioned within a blood vessel and in contact with a blood vessel wall portion in contact with the splanchnic nerve.

19. The method of claim 16, further comprising, in response to the predicted heart preload indicating that heart preload will be within the pressure range, not stimulating or blocking the splanchnic nerve.

20. The method of claim 16, wherein the sensor device is coupled to the electrical stimulation device.
